(19) United States
(12) Patent Application Publication
(10) Pub. No.: US 2020/0397492 A1
(43) Pub. Date: Dec. 24, 2020

(54) VARIABLE LENGTH SURGICAL SCREW

(71) Applicant: McGinley Engineered Solutions, LLC, Casper, WY (US)

(72) Inventors: Joseph C. McGinley, Casper, WY (US); Matthew V. Russell, Casper, WY (US); Adam M. Johnson, Casper, WY (US)

(21) Appl. No.: 16/325,850

(22) PCT Filed: Sep. 11, 2018
(86) PCT No.: PCT/US2018/050439

§ 371 Date: Feb. 15, 2019

Related U.S. Application Data
(60) Provisional application No. 62/557,430, filed on Sep. 12, 2017.

(30) Foreign Application Priority Data
Sep. 11, 2018 (US) 16325850

(51) Int. Cl. A61B 17/86

(52) U.S. Cl. CPC A61B 17/8685; A61B 2017/8655; A61B 17/8615

(57) ABSTRACT

Variable length orthopedic fasteners are described. The fasteners may include two or more distinct components which are slidingly operable to extend and/or shorten the length of the fastener. Various locking mechanisms may be provided to restrict movement of the distinct components when the fastener has been configured to a desired length. Additionally or alternatively, a variable length orthopedic fastener may be of one-piece construction. Such a fastener may comprise an engineered failure segment designed for separating the fastener into two or more pieces at a desired location.

### FIELD OF THE INVENTION

The present invention generally relates to orthopedic hardware, and in particular to screws for fixation of orthopedic hardware relative to a bone of a patient.

### BACKGROUND

In many orthopedic surgeries, various types of hardware may be affixed to other hardware or to a bone of a patient. For instance, orthopedic plates may be used for fixation of bones that have been fractured. A variety of plate types may be provided for use in different contexts. In certain instances, a plate type may be particular to a given anatomical use and/or plate function. In any regard, most often such plates are affixed to the bone of a patient using surgical fasteners such as bone screws.

In some approaches to affixing an orthopedic plate to a bone, it may be desirable to provide flexibility in relation to the length of the fastener or screw used to secure the plate to the bone. For example, it may be desirable to allow for fasteners to be inserted to various depths into the bone. It may also be desirable to use orthopedic hardware with various dimensions (e.g., thicknesses) which may require different lengths of fasteners. However, previously contemplated fasteners include drawbacks that limit the efficiency or effectiveness of such fasteners. For example, maintaining sufficient inventory of numerous sizes of fasteners may present logistical challenges which prevent all options from being made available during a surgical procedure. In this regard, only certain fasteners having a limited number of fixed lengths may be available. Alternatively, in an attempt to provide a wide variety of lengths of fasteners, a large volume of fasteners may be provided. Such a large volume of fasteners may cause delay or confusion during an operation while a surgeon or other medical staff member attempts to locate and identify a fastener having a desired length.

Additionally, with common surgical fasteners it may at times be necessary for a surgeon to utilize a fastener having a length which is longer or shorter than a desired length due to the limited availability of suitable options. In this regard, a fastener may be too short for a given application. As a consequence, the threaded portion of the fastener may fail to sufficiently engage the patient's bone to retain the orthopedic plate. Additionally or alternatively, a surgeon may over-torque such a fastener in an attempt to drive it deep enough into the bone to properly engage. This may cause the fastener to shear off, may cause the orthopedic hardware (e.g., plate) to bend, may cause the bone to fracture, or may cause metal fragments to break off and enter the surgical site.

In contrast, a surgeon may instead select a fastener which is longer than a desired length due to limited options available. In such an instance, the fastener may penetrate too deeply into the patient's bone, thereby exacerbating a fracture or causing soft tissue damage. Additionally or alternatively, a surgeon may refrain from completely inserting the fastener so as to avoid such complications. This may cause the head of the fastener to protrude from a recessed cavity in the orthopedic plate into which it is intended to be received. A protruding fastener head may cause damage to neighboring tissue or may even hinder a patient's mobility.

### SUMMARY

In view of the foregoing, improved orthopedic fasteners (i.e., screws) are needed that facilitate variability in length of the fasteners to secure an orthopedic plate to a bone of a patient. Specifically, a persistent need exists for a variable length orthopedic fastener that allows for the fastener to be used to secure a plate to a bone in a variety of applications such that the fastener may be positioned relative to the plate and bone in a manner that is functional, efficient, and safe, while penetrating the patient's bone to an appropriate depth.

Specifically, embodiments described herein may facilitate attaching an orthopedic plate to a patient's bone such that the fastener may be lengthened or shortened as needed to suit the conditions present at the time of use. In this regard, it is contemplated that a plurality of identical fasteners may be provided, and a surgeon or other user may adjust the length of each fastener as needed. Similarly, variable length fasteners having different ranges of lengths may be provided, thereby reducing the variety of fasteners needed as compared to traditional fixed length fasteners.

Various approaches to locking distinct components of a fastener together at a desired overall length are described herein. For instance, a first portion of a fastener may be configured relative to a second portion for co-rotation therewith. As an example, a fastener may comprise two members with a means of affixing one to the other. In some instances, the two members may be lockingly engaged prior to installation of any portion of the fastener into the plate or bone. For example, upon rotational advancement of one portion of a fastener, another portion may also be rotated relative to the plate to engage locking features on the fastener and/or to lockingly engage the plate to the bone. In other instances, a first portion of a fastener may be affixed to a patient's bone, and thereafter, another portion of the fastener may be attached to the first portion thereby securing the plate to the bone. For example, a portion of a fastener may be independently rotatable relative to another portion. As such, once a first portion of the fastener is advanced into the bone, a second portion may then be independently rotated or affixed to the first portion to engage a locking mechanism between the first portion and the second portion.

A first aspect may comprise an orthopedic fastener for use in securing an orthopedic plate to a bone of a patient. The fastener may include a post and a stud. The post may include a head portion having a first recess at a proximal end of the post for rotational engagement of the post by a tool (e.g., drill, bit driver, screw driver, etc.) and an elongate portion on a distal portion of the post. A first bore may extend along a central axis of the elongate portion from a distal end of the post toward the head portion. The first bore may be defined by a threaded wall. The stud may include first threads for engagement with a bone of a patient at a distal portion and second threads at a proximal portion for engagement with the threaded wall of the first bore. The stud may further include a second recess at a proximal end for rotational engagement of the stud. In this regard, the stud may be driven independently of the post. For example, the stud may be driven into the bone of a patient prior to attachment of the post to the stud. Additionally or alternatively, a second bore may extend from a distal end of the first recess on the head portion of the post into a proximal end of the first bore to allow a tool to be inserted through the first bore to engage the second recess of the stud after the post has been affixed to the stud.

A number of feature refinements and additional features are applicable to the first aspect. These feature refinements and additional features may be used individually or in any combination. As such, each of the following features that will be discussed may be, but are not required to be, used with any other feature or combination of features of the first aspect.

For instance, in an embodiment, the first threads of the stud of a fastener (e.g., those for engagement with the bone of a patient) may be disposed around a first cylinder having a first diameter. The second threads (e.g., those for engagement with the post) may be disposed around a second cylinder having a second diameter. The second diameter may correspond to a diameter of the first bore for engagement of the second threads with the threaded wall of the first bore. In some embodiments, the first diameter may be greater than the second diameter while in other embodiments, the first diameter may be less than or equal to the second diameter.

In some embodiments, a diameter of the head portion may exceed a maximum diameter of the stud and a maximum diameter of the elongate portion of the post. In this regard, the stud and elongate portion of the post may pass freely through an opening in an orthopedic plate while the head portion may be larger than, and therefore engage, the orthopedic plate. Alternatively, the first threads of a stud may have a larger diameter than an opening in an orthopedic plate. In this regard, the orthopedic plate may be retained between the first threads of the stud and the head portion of the post when the stud and post are engaged.

In a second aspect, an orthopedic fastener for use in securing an orthopedic plate to a bone of a patient may include a head portion and an elongate portion. The head portion may be disposed at a proximal portion of the fastener and may have a recess at a proximal end of the head portion for rotational engagement of the fastener. The elongate portion may be disposed at a distal portion of the fastener. The elongate portion may include a first thread configured for engagement with a portion of a bone of a patient and a second thread configured for engagement with a portion of a bone of a patient. An engineered failure segment may be disposed between the first thread and the second thread configured to structurally fail upon subjection to a failure force (e.g., failure torsional force, failure tension stress, failure shear force, etc.). In this regard, a surgeon or other user may exert a force upon the fastener equal to or exceeding the failure force to detach a portion of the elongate portion from the remainder of the fastener. The failure force may be exerted manually, by hand or with a tool (e.g., pliers, nibbler shears, etc.), or may be exerted automatically with a powered cutting tool. The failure force may have a magnitude which is greater than a magnitude of a maximum expected force associated with an installation procedure of the fastener. In this regard, a fastener may be resistant to undesired separation during use. In some instances, an engineered failure segment may be cut with a saw, rotary tool, etc. rather than snapped apart.

Additionally, a plurality of engineered failure segments may be provided along the length of the elongate portion. For example, one engineered failure segment may be disposed between each pair of adjacent threads. In this regard, a surgeon or other user may select an engineered failure segment corresponding to a desired resultant length of the fastener. By snapping or otherwise separating the fastener at the selected engineered failure segment, the user may alter the length of the fastener as desired.

A number of feature refinements and additional features are applicable to the second aspect. These feature refinements and additional features may be used individually or in any combination. As such, each of the following features that will be discussed may be, but are not required to be, used with any other feature or combination of features of the second aspect. Moreover, any of the foregoing features or feature refinements described in relation to the first aspect may be utilized in any combination with the second aspect.

For example, in an embodiment, the first thread may be disposed upon a first segment of the elongate portion having a first diameter. The second thread may be disposed upon a second segment of the elongate portion having the first diameter and the engineered failure segment may comprise a portion of the elongate portion having a second diameter which is smaller than the first diameter. In this regard, the fastener may be biased to structurally fail at the location of the engineered failure segment due to a reduced cross-sectional area.

In yet another embodiment, the first thread may be disposed upon a first segment of the elongate portion comprising a first material composition while the second thread may be disposed upon a second segment of the elongate portion having the first material composition. The engineered failure segment may comprise a portion of the elongate portion having a second material composition (e.g., aluminum) which has a lesser shear strength than first material composition (e.g., steel). In this regard, the fastener may be biased to structurally fail at the location of the engineered failure segment due to a reduced shear strength of the second material composition. In a fastener utilizing disparate material compositions, the diameter of the elongate portion at the engineered failure segment may or may not be smaller than a maximum diameter of the elongate portion. Utilization of both a smaller diameter and a weaker material composition at the engineered failure segment may increase the likelihood of failure at the desired location.

In a third aspect, an orthopedic fastener for use in securing an orthopedic plate to a bone of a patient may include a post, a stud, and a locking mechanism. The post may include a head portion having a first recess at a proximal end and an elongate portion on a distal portion of the post. A bore may extend along a central axis of the elongate portion from a distal end thereof toward the head portion. The bore may extend into the first recess or may be offset therefrom along the central axis. Additionally or alternatively, an inner wall defining the bore may be smooth, textured, or threaded as appropriate for engagement of the stud. The stud may have a threaded portion for engagement with a bone of a patient at a distal portion and a shaft extending from a proximal end of the distal portion. The shaft may be sized to be receivable within the bore of the post. A locking mechanism may secure the post to the stud in fixed relation.

A number of feature refinements and additional features are applicable to the third aspect. These feature refinements and additional features may be used individually or in any combination. As such, each of the following features that will be discussed may be, but are not required to be, used with any other feature or combination of features of the third aspect. Moreover, any of the foregoing features or feature refinements described in relation to the first and second aspects may be utilized in any combination with the third aspect.

In an embodiment, the locking mechanism may comprise a locking screw and a corrugated portion of the stud. Although the corrugated portion as described in relation to this embodiment is contemplated as including ridges and grooves, it is also contemplated that the surfaces may be smooth, may have barbs, may be coated with a polymer or rubber, or may have some other means of frictional or interlocking engagement with an interior wall of the bore of the post. Corrugations are only one of many means contemplated for generating frictional engagement.

The locking screw may include a recess at a proximal end for rotational engagement of the locking screw and a threaded portion. The threads disposed adjacent to a distal end of the threaded portion may have diameters which are smaller than threads disposed adjacent a proximal end of the threaded portion. In this regard, the locking screw may be tapered. Additionally or alternatively, a threaded bore configured for receipt of the locking screw may be tapered to have a smaller diameter at a distal end relative to a proximal end.

The post may further include a second bore extending along the central axis from a distal end of the first recess to a proximal end of the first bore. The second bore may be sized for receipt of a tool configured to engage the recess of the locking screw. The corrugated portion of the stud may be disposed near a proximal end of the shaft and comprise a plurality of fingers sized for receipt within the first bore of the post. The exterior surfaces (i.e., those disposed furthest from the central axis) may comprise at least a portion of the corrugated portion. The corrugated portion may further comprise the threaded bore extending from a proximal end of the stud and sized for receipt of the locking screw such that rotational driving of the locking screw into the threaded bore causes proximal ends of the fingers to spread radially apart from one another to engage the interior wall of the first bore of the post.

In another embodiment, a post may further comprise a slot extending from a distal end of the post toward the proximal end of the post. The stud may comprise a ridge along a side of the shaft that is slidably receivable within the slot. In this regard, rotation of the post may cause the slot to rotate around the central axis. Rotation of the slot, in turn, may engage the ridge extending from the shaft of the stud to rotationally engage the stud. In this regard, rotation of the post may cause rotation of the stud, thereby driving the stud into the bone of a patient.

In another embodiment, a locking mechanism may comprise a plurality of ribs disposed along the shaft of the stud and a clip configured to engage the ribs. The clip may be removably disposable within a window passing through a wall of the first bore such that one or more ribs of the clip may engage at least one corresponding rib of the shaft to lockingly engage the stud to prevent longitudinal movement along the central axis relative to the post.

In another embodiment, a locking mechanism may comprise a plurality of apertures disposed along the ridge of the stud. The locking mechanism may further comprise at least one aperture disposed through a wall of the first bore and a locking pin slidably disposable within one aperture of the plurality of apertures disposed along the ridge of the stud and the at least one aperture disposed through the wall of the first bore. The locking pin may be operable to restrict movement of the stud relative to the post.

In another embodiment, a locking mechanism may comprise a plurality of barbs disposed upon an external surface of the shaft of the stud. The barbs may be operable to engage the inner wall of the bore extending from the distal end of the post, and/or to engage a distal surface of the post to restrict movement of the post in a direction toward the threaded portion of the stud which would tend to shorten the length of the fastener. Additionally or alternatively, the stud may comprise a bore and the post may comprise a shaft configured for receipt therein.

In another embodiment, the locking mechanism may comprise at least one aperture disposed through a wall of the elongate portion of the post. The locking mechanism may further comprise at least one dimple disposed on an external surface of the shaft of the stud and a set screw disposable within the at least one aperture through the wall of the post. The set screw may be operable to engage the at least one dimple to restrict longitudinal movement of the stud along the central axis relative to the post.

Alternatively, the set screw may be omitted in some instances of this embodiment. In such an instance, a crimping tool or similar device may be at least partially inserted through the at least one aperture to engage a dimple disposed on the external surface of the shaft. In this regard, the shaft may be comprised of a material designed to yield under a crimping force exerted by the crimping tool. Yielding of the shaft may cause deformation which permits the shaft to frictionally engage the inner wall of the post, thereby restricting longitudinal movement of the stud along the central axis relative to the post. Whether the locking mechanism comprises a set screw and/or crimping, the dimples may be optionally omitted.

A fourth aspect includes a method of use of an orthopedic fastener. The method includes placing a plate body that extends between an upper surface and a lower surface relative to a bone of a patient such that the lower surface is in contacting engagement with the bone of the patient. The plate body may comprise an aperture extending through the plate body from the upper surface to the lower surface along a reference axis. The method also includes manipulating a first member of a fastener relative to a second member of the fastener to configure the fastener to a desired length, and engaging a locking mechanism of the fastener. The method further includes advancing at least a portion of the fastener through the aperture.

A fifth aspect includes a method of use of an orthopedic fastener. The method includes placing a plate body that extends between an upper surface and a lower surface relative to a bone of a patient such that the lower surface is in contacting engagement with the bone of the patient. The plate body may comprise an aperture extending through the plate body from the upper surface to the lower surface along a reference axis. The method also includes exerting a force on a fastener having a distal portion and a proximal portion, the force sufficient to detach the distal portion of the fastener to reduce a length of the fastener to a desired length. The method further includes advancing at least a portion of the proximal portion of the fastener through the aperture.

### BRIEF DESCRIPTION OF THE DRAWINGS

FIG. 1A is a perspective view of an embodiment of a stud as may be used in a variable length orthopedic fastener.

FIG. 1B is a perspective view of an embodiment of a post and a stud as seen in FIG. 1A forming a variable length orthopedic fastener.

FIG. 2 is a perspective view of an embodiment of a variable length orthopedic fastener having a designed failure plane.

FIG. 3 is a perspective view of an embodiment of a variable length orthopedic fastener having a locking mechanism comprising a clip disposable within a window to engage ribs on the shaft.

FIG. 4 is a perspective view of an embodiment of a variable length orthopedic fastener having a locking mechanism comprising apertures and a locking pin.

FIG. 5 is a perspective view of an embodiment of a variable length orthopedic fastener having a locking mechanism comprising barbs.

FIG. 6 is a perspective view of an embodiment of a variable length orthopedic fastener having a locking mechanism comprising dimples and an aperture for engagement with a crimping tool.

FIG. 7 is a perspective view of an embodiment of a variable length orthopedic fastener having a locking mechanism comprising a locking screw for radial manipulation of a plurality of fingers.

### DETAILED DESCRIPTION

The following description is not intended to limit the invention to the forms disclosed herein. Consequently, variations and modifications commensurate with the following teachings, skill and knowledge of the relevant art, are within the scope of the present invention. The embodiments described herein are further intended to explain modes known of practicing the invention and to enable others skilled in the art to utilize the invention in such, or other embodiments and with various modifications required by the particular application(s) or use(s) of the present invention.

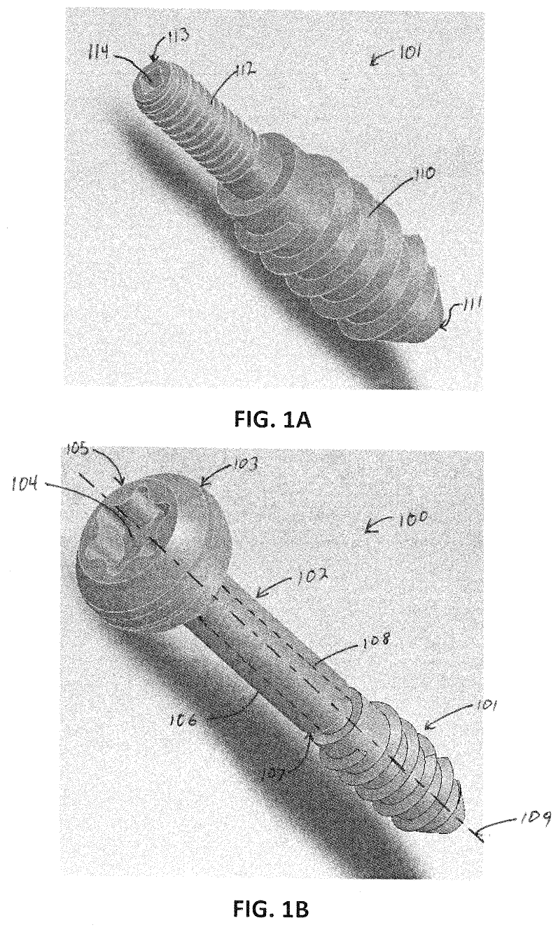

FIG. 1A is a perspective view of an embodiment of a stud 101 as may be used in a variable length orthopedic fastener. The stud 101 may include first threads 110 for engagement with a bone of a patient at a distal portion 111 and second threads 112 at a proximal portion 113 for engagement with a post (see, e.g., FIG. 1B). The stud 101 may further include a recess 114 at a proximal end 113 for rotational engagement of the stud 101.

FIG. 1B is a perspective view of an embodiment of a post 102 and a stud 101, as seen in FIG. 1A, forming a variable length orthopedic fastener 100. The post 102 may include a head portion 103 having a first recess 104 at a proximal end 105 of the post 102 for rotational engagement of the post 102 by a tool (e.g., bit, driver, etc.). The post 102 may also include an elongate portion 106 on a distal portion 107 of the post 102. A first bore 108 may extend along a central axis 109 of the elongate portion 106 from a distal end 107 of the post toward the head portion 103. The first bore 108 may be defined by a threaded interior wall having threads corresponding to the second threads 112 of the stud. Alternatively, the interior wall of the first bore 108 may be smooth or otherwise textured.

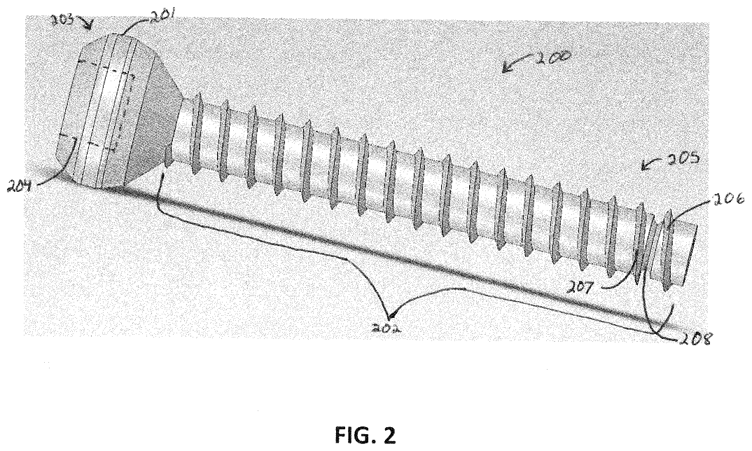

FIG. 2 is a perspective view of an embodiment of a variable length orthopedic fastener 200 having a designed failure plane. The fastener 200 may include a head portion 201 and an elongate portion 202. The head portion 201 may be disposed at a proximal portion 203 of the fastener and may have a recess 204 at a proximal end of the head portion for rotational engagement of the fastener 200 by a tool. The elongate portion 202 may be disposed at a distal portion 205 of the fastener 200. The elongate portion 202 may include a first thread 206 and a second thread 207 for engagement with a portion of a bone of a patient. An engineered failure segment 208 comprising a designed failure plane may be disposed between the first thread 206 and the second thread 207 and configured to structurally fail upon subjection to a failure force.

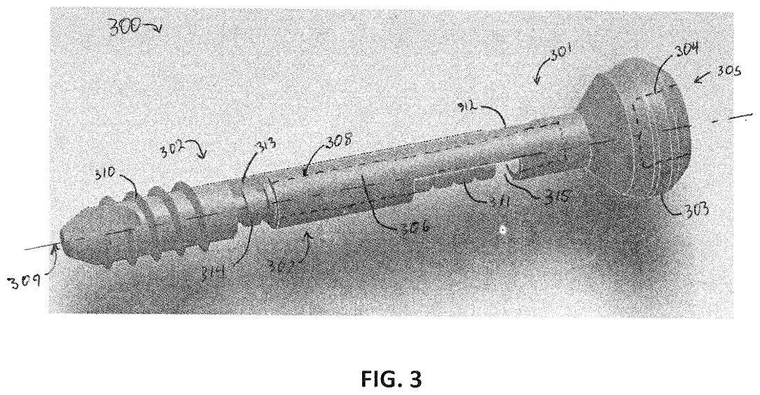

FIG. 3 is a perspective view of an embodiment of a variable length orthopedic fastener 300 having a locking mechanism comprising a clip (not shown) disposable within a window 315 to engage ribs 314 on a shaft 311. A post 301 may include a head portion 303 having a first recess 304 at a proximal end 305 and an elongate portion 306 on a distal portion 307 of the post 301. A bore 308 may extend along a central axis 309 of the elongate portion 306 from a distal end thereof toward the head portion 303. A stud 302 may have a threaded portion 310 for engagement with a bone of a patient at a distal portion and a shaft 311 extending from a proximal end of the distal portion. The shaft 311 may be sized to be receivable within the bore 308 of the post 301. The post 301 may further comprise a slot 312 extending from a distal end of the post 301 toward the proximal end of the post 301. The stud 302 may comprise a ridge 313 along a side of the shaft 311 that is slidably receivable within the slot 312. The locking mechanism may comprise a plurality of ribs 314 disposed along the shaft 311 and a clip (not shown). The clip may be removably disposable within a window 315 passing through a wall of the first bore 308 such that one or more ribs of the clip engage the ribs 314 of the shaft 311. The clip may be configured for receipt within and/or engagement with the window 315. For example, the clip may have the same dimensions (i.e., width, height, thickness, or curvature) as the window 315 for secure fitment therein. Alternatively, the clip may be a substantially flat C-shaped clip that snaps around the shaft 311 between adjacent ribs 314. In this regard, the C-shaped clip may extend into the open space of the window 315 and may engage a perimeter of the window 315 to prevent movement of the stud 302 with respect to the post 301.

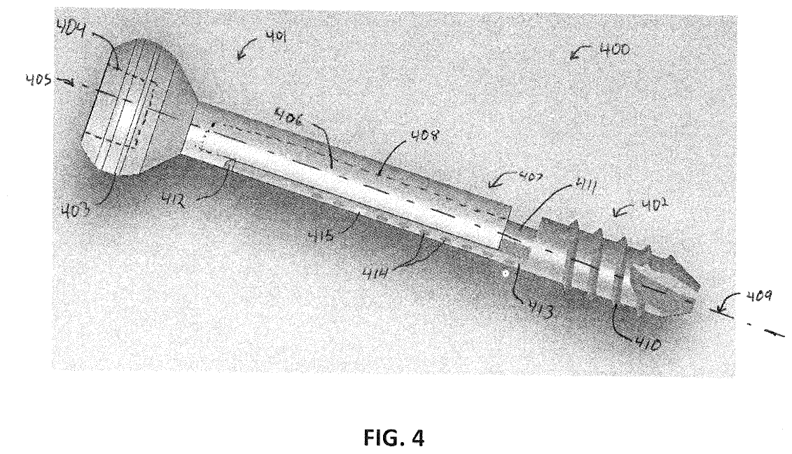

FIG. 4 is a perspective view of an embodiment of a variable length orthopedic fastener 400 having a locking mechanism comprising apertures 414 and a locking pin 415. A post 401 may include a head portion 403 having a first recess 404 at a proximal end 405 and an elongate portion 406 on a distal portion 407 of the post 401. A bore 408 may extend along a central axis 409 of the elongate portion 406 from a distal end thereof toward the head portion 403. A stud 402 may have a threaded portion 410 for engagement with a bone of a patient at a distal portion and a shaft 411 extending from a proximal end of the distal portion. The shaft 411 may be sized to be receivable within the bore 408 of the post 401. The post 401 may further comprise a slot 412 extending from a distal end of the post 401 toward the proximal end of the post 401. The stud 402 may comprise a ridge 413 along a side of the shaft 411 that is slidably receivable within the slot 412. A locking mechanism may comprise a plurality of apertures 414 disposed along the ridge 413 of the stud 402. The locking mechanism may further comprise at least one aperture (not shown) disposed through a wall of the first bore 408 and a locking pin 415 slidably disposable within one aperture of the plurality of apertures 414 disposed along the ridge 413 of the stud 402 and the at least one aperture disposed through, or recess disposed in, the wall of the first bore 408. The locking pin 415 may be operable to restrict movement of the stud 412 relative to the post 401. Notably, a ridge 413 and slot 412 may be omitted from such a fastener 400. In such an instance, the locking pin 415 may serve to restrict longitudinal and rotational movement of the stud 402 with respect to the post 401.

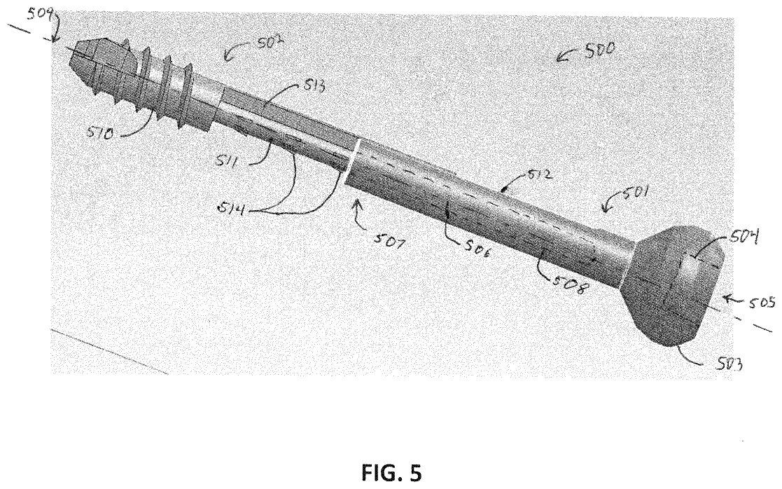

FIG. 5 is a perspective view of an embodiment of a variable length orthopedic fastener 500 having a locking mechanism comprising barbs 514. A post 501 may include a head portion 503 having a first recess 504 at a proximal end 505 and an elongate portion 506 on a distal portion 507 of the post 501. A bore 508 may extend along a central axis 509 of the elongate portion 506 from a distal end thereof toward the head portion 503. A stud 502 may have a threaded portion 510 for engagement with a bone of a patient at a distal portion and a shaft 511 extending from a proximal end of the distal portion. The shaft 511 may be sized to be receivable within the bore 508 of the post 501. The post 501 may further comprise a slot 512 extending from a distal end of the post 501 toward the proximal end of the post 501. The stud 502 may comprise a ridge 513 along a side of the shaft 511 that is slidably receivable within the slot 512. A locking mechanism may comprise a plurality of barbs 514 disposed upon an external surface of the shaft 511. The barbs 514 may be operable to engage a wall defining the bore 508 in the post 501 to restrict movement of the post 501 in a direction toward the threaded portion 510 of the stud 502. The barbs 514 may be retractable or flexible so as to fold toward the shaft 511 when the barbs 514 are disposed within the bore 508. Such barbs 514 may be mechanically biased to protrude outwardly away from the central axis 509 when disposed outside the bore 508. Fastener 500 may be provided with shaft 511 fully inserted into bore 508. In this regard, a user may manipulate the fastener 500 by pulling the stud 502 partially out of the bore 508 to extend the length of the fastener 500.

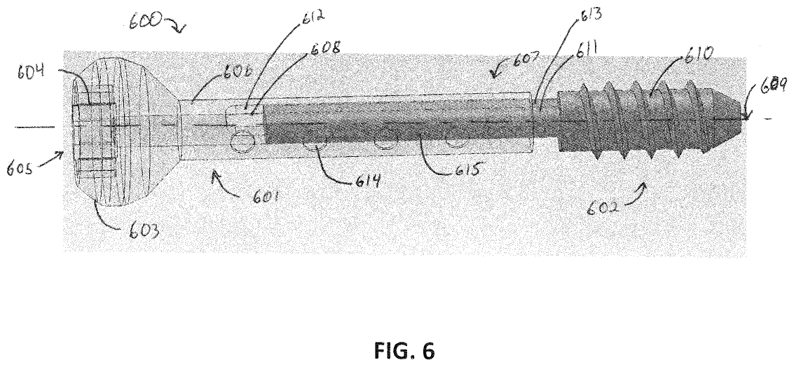

FIG. 6 is a perspective view of an embodiment of a variable length orthopedic fastener 600 having a locking mechanism comprising dimples 615 and an aperture (a set screw may be utilized although not shown in the illustrated embodiment). A post 601 may include a head portion 603 having a first recess 604 at a proximal end 605 and an elongate portion 606 on a distal portion 607 of the post 601. A bore 608 may extend along a central axis 609 of the elongate portion 606 from a distal end thereof toward the head portion 603. A stud 602 may have a threaded portion 610 for engagement with a bone of a patient at a distal portion and a shaft 611 extending from a proximal end of the distal portion. The shaft 611 may be sized to be receivable within the bore 608 of the post 601. The post 601 may further comprise a slot 612 extending from a distal end of the post 601 toward the proximal end of the post 601. The stud 602 may comprise a ridge 613 along a side of the shaft 611 that is slidably receivable within the slot 612. A locking mechanism may comprise at least one aperture 614 disposed through a wall of the elongate portion 606 of the post 601 into the bore 608. In embodiments utilizing a set screw, the aperture 614 may be defined by a threaded surface for engagement with threads of the set screw. The locking mechanism may further comprise at least one dimple 615 disposed on an external surface of the shaft 611 which is designed to be engaged by a crimping tool (not shown) disposed through the at least one aperture 614. The at least one dimple 615 may be configured to promote deformation of the shaft to engage an inner surface of the post 602 to restrict movement of the stud 602 relative to the post 601. In embodiments utilizing crimping, the at least one aperture 614 may not include threads. In some embodiments, both crimping and a set screw may be used.

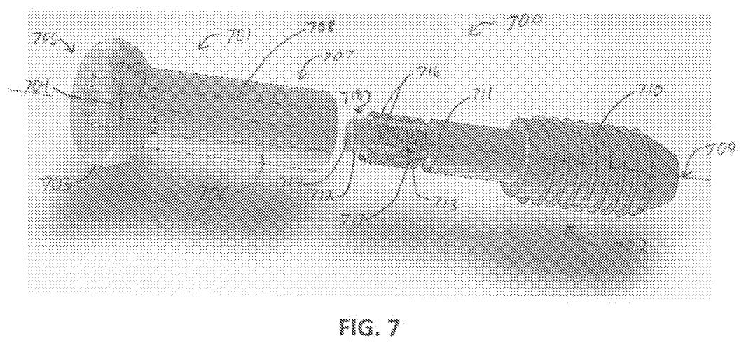

FIG. 7 is a perspective view of an embodiment of a variable length orthopedic fastener 700 having a locking mechanism comprising a locking screw 718 for radial manipulation of a plurality of fingers 716. A post 701 may include a head portion 703 having a first recess 704 at a proximal end 705 and an elongate portion 706 on a distal portion 707 of the post 701. A bore 708 may extend along a central axis 709 of the elongate portion 706 from a distal end thereof toward the head portion 703. A stud 702 may have a threaded portion 710 for engagement with a bone of a patient at a distal portion and a shaft 711 extending from a proximal end of the distal portion. The shaft 711 may be sized to be receivable within the bore 708 of the post 701. A locking mechanism may comprise a locking screw 718 and a corrugated portion 713 of the stud 702. The locking screw 718 may include a recess 714 at a proximal end for rotational engagement of the locking screw 718 by a tool and a threaded portion 712. Threads disposed adjacent to a distal end of the threaded portion 712 may have diameters which are smaller than diameters of threads disposed adjacent a proximal end of the threaded portion 712. The post 701 may further include a second bore 715 extending along the central axis 709 from a distal end of the first recess 704 to a proximal end of the first bore 708. The corrugated portion 713 of the stud 702 may be disposed near a proximal end of the shaft 711 and comprise a plurality of fingers 716 initially configured (sized and positioned) for receipt within the first bore 708 of the post 701 when the locking screw 718 is at least partially retracted from the stud 702. The corrugated portion 713 may further comprise a threaded bore 717 extending from a proximal end of the stud 702 sized for receipt of the locking screw 718 such that rotational driving of the locking screw 718 into the threaded bore 717 causes proximal ends of the fingers 716 to spread apart from one another to engage a wall of the first bore 708 of the post 701. In some embodiments, the fingers 716 may be mechanically biased to retract toward the central axis 709 when the locking screw 218 is retracted.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain known modes of practicing the invention and to enable others skilled in the art to utilize the invention in such or other embodiments and with various modifications required by the particular application(s) or use(s) of the present invention. It is intended that the appended claims be construed to include alternative embodiments to the extent permitted by the prior art.

1-11. (canceled)

12. An orthopedic fastener for use in securing an orthopedic plate to a bone of a patient, the orthopedic fastener comprising:
a post comprising a head portion having a first recess at a proximal end and an elongate portion on a distal portion of the post, wherein a bore extends along a central axis of the elongate portion from a distal end thereof toward the head portion;
a stud comprising a threaded portion for engagement with a bone of a patient at a distal portion of the stud and a shaft extending from a proximal end of the distal portion, the shaft sized to be receivable within the bore of the post, and
a locking mechanism for securing the post to the stud in fixed relation, wherein the locking mechanism comprises a locking screw and a corrugated portion of the stud, wherein:
the locking screw comprises:
a recess at a proximal end for rotational engagement of the locking screw; and
a threaded portion wherein threads disposed adjacent a distal end of the threaded portion have diameters which are smaller than threads disposed adjacent a proximal end of the threaded portion;
the post further comprises a second bore extending along the central axis from a distal end of the first recess to a proximal end of the first bore; and
the corrugated portion of the stud is disposed near a proximal end of the shaft, the corrugated portion comprises a plurality of fingers configured for receipt within the first bore of the post, the corrugated portion further comprising a threaded bore extending from a proximal end of the stud and sized for receipt of the locking screw, wherein rotational driving of the locking screw into the threaded bore causes proximal ends of the fingers to spread apart from one another to engage a wall of the first bore of the post.

13. An orthopedic fastener for use in securing an orthopedic plate to a bone of a patient, the orthopedic fastener comprising:
a post comprising a head portion having a first recess at a proximal end and an elongate portion on a distal portion of the post, wherein a bore extends along a central axis of the elongate portion from a distal end thereof toward the head portion;
a stud comprising a threaded portion for engagement with a bone of a patient at a distal portion of the stud and a shaft extending from a proximal end of the distal portion, the shaft sized to be receivable within the bore of the post; and
a locking mechanism for securing the post to the stud in fixed relation, wherein the post further comprises a slot extending from a distal end of the post toward the proximal end of the post, and wherein the stud comprises a ridge along a side of the shaft, the ridge being slidably receivable within the slot.

14. The orthopedic fastener of claim 13, wherein the locking mechanism comprises:
a plurality of ribs disposed along the shaft; and
a clip, wherein the clip is removably disposable within a window passing through a wall of the first bore such that the clip engages the ribs of the shaft and a portion of the wall of the first bore defining the window to restrict movement of the post relative to the stud.

15. The orthopedic fastener of claim 13, wherein the locking mechanism comprises:
a plurality of apertures disposed along the ridge of the stud;
at least one aperture disposed through a wall of the first bore; and
a locking pin slidably disposable within an aperture of the plurality of apertures disposed along the ridge of the stud and the at least one aperture disposed through the wall of the first bore, the locking pin operable to restrict movement of the stud relative to the post.

16. The orthopedic fastener of claim 13, wherein the locking mechanism comprises:
a plurality of apertures disposed along the ridge of the stud;
at least one recess disposed on a wall of the first bore; and
a locking pin slidably disposable within an aperture of the plurality of apertures disposed along the ridge of the stud and the at least one recess, the locking pin operable to restrict movement of the stud relative to the post.

17. The orthopedic fastener of claim 13, wherein the locking mechanism comprises a plurality of barbs disposed upon an external surface of the shaft, wherein the barbs are operable to engage the distal end of the post to restrict movement of the post in a direction toward the threaded portion of the stud.

18. The orthopedic fastener of claim 13, wherein the locking mechanism comprises:
at least one aperture disposed through the elongate portion of the post; and
at least one dimple disposed on an external surface of the shaft, wherein a crimping tool is at least partially disposable within the at least one aperture to engage the at least one dimple to deform at least a portion of the shaft to engage an inner wall of the bore to restrict movement of the stud relative to the post.
